(19) United States
(12) Patent Application Publication
(10) Pub. No.: US 2025/0082440 A1
(43) Pub. Date: Mar. 13, 2025

(54) Composition for Isolating Tissue

(71) Applicant: 3M INNOVATIVE PROPERTIES COMPANY, St. Paul, MN (US)

(72) Inventors: Henning Hoffmann, Windach (DE); Peter Osswald, Tuerkheim (DE); Ruediger Hampe, Puergen (DE)

(21) Appl. No.: 18/580,190

(22) PCT Filed: Jul. 18, 2022
(86) PCT No.: PCT/IB2022/056602

§ 371 Date: Jan. 18, 2024

(30) Foreign Application Priority Data
Aug. 5, 2021 (EP) 21189752.5

(51) Int. Cl. A61C 5/82; A61L 31/04

(52) U.S. Cl. CPC A61C 5/82; A61L 31/041

(57) ABSTRACT

The invention relates to a composition for isolating tissue, the composition comprising a radiation curable component A1, preferably being present in an amount of at least 40 wt. % with respect to the composition, the radiation curable component A1 comprising a polyether polyol spacer group having a molecular weight (Mw) of 1,000 to 20,000 g/mol, and at least two (meth)acrylate moieties, photo-initiator, guanidyl containing polymer, carrageenan, optionally softener, optionally dye, optionally filler, optionally additives. The invention also relates to a process for temporarily isolating tissue, the process comprising the steps of providing the composition described in any of the preceding claims, placing the composition in contact with tissue, radiation curing the composition at least partially for a time period T1, optionally moving or adjusting the partially cured composition, optionally radiation curing the partially cured composition for a time period T2, wherein T2>T1, removing the composition from the tissue.

### FIELD OF THE INVENTION

The invention relates to a composition with liquid-absorbing properties for isolating tissue. The composition can be used as a curable alternative to a dental rubber dam.

The invention also relates to a process for temporarily isolating tissue, wherein the composition with liquid-absorbing properties is at least partially cured by radiation.

### BACKGROUND

In many dental procedures an isolation of the working field against liquids from the oral cavity such as saliva or blood is needed.

Usually a rubber dam is used to protect the tooth to be treated from liquids that may negatively impact the properties of dental material such as adhesives or cements. These rubber dams are often pretty cumbersome to apply and are therefore not suitable in all cases. Especially the sealing of dental matrices which are sometimes used in the filling therapy can be challenging to the practitioner. Sometimes already a local isolation of the tissue is sufficient, in particular if only small quantities of dental adhesives, dental cements or dental filling materials are applied.

Curable liquid alternatives are known but not widely spread.

U.S. Pat. No. 5,098,299 (Fischer) describes a composition for repairing and sealing dental dams in the mouth of a patient, the composition comprising a silicone-based material in an amount of 15 to 60 wt. %, a lower weight aliphatic glycol in an amount up to 60 wt. % and a cellulosic material in an amount of 10 to 60 wt. %.

U.S. Pat. No. 6,305,936 B1 (Jensen et al.) relates to a polymerizable dental isolation barrier for isolating a dental substrate comprising a monomer (e.g. polyethylene glycol dimethacrylate or urethane methacrylate), a curing agent and an organic polymerization strength reducer (e.g. a polyol).

U.S. Pat. No. 7,157,502 B2 (Stannard) describes a method for forming a polymerizable dental barrier material about dental tissue in the oral cavity of a patient. The dental barrier material comprises a polymerization system and a polymer with reactive end groups having a molecular weight of more than 20,000 g/mol and being present in a concentration of 50 to 90 wt. %.

U.S. Pat. No. 7,789,662 B2 (Van Eikeren et al.) relates to a dental masking product for teeth and gum which cross-links in a self-curing manner in the mouth on the gingiva and produces an elastomeric material, wherein the masking product comprises an A-silicone, C-silicone or polyether.

U.S. Pat. No. 10,751,264 B2 (Craig et al.) relates to a curable composition and methods for isolating a working area. The curable composition includes a borate-crosslinked polysiloxane, an ethylenically unsaturated monomer with two polymerizable groups and an initiator.

US 2011/0046262 A1 (Bublewitz et al.) describes a pasty insert material for widening of gingival sulcus, which contains a paste-forming agent, a particulate superabsorber and an astringent.

US 2010/0248190 A1 (Chen et al.) describes a method for temporarily widening a gingival sulcus using an uncured composition comprising a polymerizable monomer, a photo polymerization initiator, a fine inorganic powder and an astringent. The uncured composition is said to have a viscosity that is higher than 13,000 Pa*s.

US 2019/0282453 A1 (Hoffmann et al.) relates to a medical composition containing guanidinyl-containing polymers and carrageenan. The composition is useful for absorbing water-containing fluids and be used as dental retraction composition and is able to keep a sulcus of a tooth open.

### SUMMARY OF INVENTION

There is need for a dental composition useful for isolating or protecting tissue in the mouth of a patient, which is sufficiently hydrophilic and able to keep the preparation zone or working zone sufficiently dry.

Ideally, the dental composition can be removed from the working area easily after use and if possible in one piece. The composition should be sufficiently hydrophilic and elastic to be able to adhere even to wet tissue. It would also be beneficial, if the composition can be applied to the working area easily and moved or shaped after application closer to the preparation zone, if desired.

In one embodiment the present invention features a composition for isolating tissue composition for isolating tissue, the composition comprising a radiation curable component preferably in an amount of at least 40 wt. % with respect to the composition, the radiation curable component comprising a polyether polyol spacer group having a molecular weight Mw of 1,000 to 20,000 g/mol, and at least two (meth)acrylate moieties, photo-initiator, guanidyl containing polymer, carrageenan, optionally softener, optionally dye, optionally filler, optionally additives, the composition having preferably a viscosity of less than 200 Pa*s at 23° C. and a shear rate of 50s⁻¹.

The invention also relates to a process of temporarily isolating tissue, the process comprising the steps of providing the composition described in present text and claims, placing the composition in contact with tissue, radiation curing the composition at least partially for a time period T1, optionally moving or adjusting the partially cured composition, optionally radiation curing the partially cured composition for a time period T2, wherein T2>T1, removing the composition from the tissue.

A further embodiment of the invention is directed to a kit of parts comprising the composition described in the present text and claims and the following items alone or in combination: dental curing light, dental restoration material, dental adhesive, dental cement, dental matrix equipment.

Unless defined differently, for this description the following terms shall have the given meaning:

The term “compound” or “component” is a chemical substance which has a certain molecular identity or is made of a mixture of such substances, e.g., polymeric substances.

A “hardenable or curable or polymerizable component” is any component which can be cured or solidified in the presence of a photo-initiator by radiation-induced polymerization. A hardenable component may contain only one, two, three or more polymerizable groups. Typical examples of polymerizable groups include unsaturated carbon groups, such as a vinyl group being present i.a. in a (methyl)acrylate group.

As used herein, “(meth)acryl” is a shorthand term referring to “acryl” and/or “methacryl”. For example, a “(meth)acryloxy” group is a shorthand term referring to either an acryloxy group (i.e., CH₂═CH—C(O)—O—) and/or a methacryloxy group (i.e., CH₂═C(CH₃)—C(O)—O—).

As used herein, “hardening” or “curing” a composition are used interchangeably and refer to polymerization and/or crosslinking reactions including, for example, photo-polymerization reactions and chemical-polymerization techniques (e.g., ionic reactions or chemical reactions forming radicals effective to polymerize ethylenically unsaturated compounds) involving one or more materials included in the composition.

A “photo-initiator” is a substance being able to start or initiate the curing process of a hardenable composition in the presence of radiation, in particular light having a wavelength in the range of 300 to 700 nm, in particular 430 to 700 nm.

“One component composition” means that all of the components mentioned are present in the composition during storage and use. That is, the composition to be applied or used is not prepared by mixing different parts of the composition before use. In contrast to one-component compositions, those compositions are often referred to as two-component compositions (e.g. being formulated as powder/liquid, liquid/liquid or paste/paste compositions). The composition for isolating tissue described in the present text is a one-component composition.

“Dental restoration” means dental articles which are used for restoring a tooth to be treated. Examples of dental restorations include crowns, bridges, inlays, onlays, veneers, facings, copings, crown and bridged framework, and parts thereof.

A “water or liquid absorbing component” is a component being able to absorb water in an amount of at least 50 wt. % or at least 100 wt. % or at least 200 wt. % with respect to the weight of the component.

A “tooth structure” is any tooth structure, prepared or ready for preparation by the dentist. It can be a single tooth or two or more teeth. A tooth structure is also referred to as hard dental tissue in contrast to soft dental tissue (e.g. gingiva).

By “paste” is meant a soft, viscous mass of solids (i.e. particles) dispersed in a liquid.

A “particle” means a substance being a solid having a shape which can be geometrically determined. The shape can be regular or irregular. Particles can typically be analysed with respect to e.g. particle size and particle size distribution.

The particle size (d50) of a powder can be obtained from the cumulative curve of the grain size distribution. Respective measurements can be done using commercially available granulometers (e.g. Malvern Mastersizer 2000). “D” represents the diameter of powder particles and “50” refers to the volume percentage of the particles. Sometimes, the 50% is also expressed as “0.5”. For example, “(d50)=1 μm” means that 50% of the particles have a size of 1 μm or less.

“Radiation curable” shall mean that the component (or composition, as the case may be) can be cured by applying radiation, preferably electromagnetic radiation with a wavelength in the visible light spectrum under ambient conditions and within a reasonable time frame (e.g. within about 60, 30 or 10 seconds).

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar (at maritime level).

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

A composition is “essentially or substantially free of” a certain component, if the composition does not contain said component as an essential feature. Thus, said component is not willfully added to the composition either as such or in combination with other components or ingredient of other components. A composition being essentially free of a certain component usually does not contain that component at all. However, sometimes the presence of a small amount of the said component is not avoidable e.g. due to impurities contained in the raw materials used.

### BRIEF DESCRIPTION OF FIGURES

FIGS. 1-4 exemplify how the composition described in the present text can be applied, cured and removed by the practitioner.

FIGS. 5-10 exemplify an alternative process including the curing of the composition described in the present text in two steps.

### DETAILED DESCRIPTION

It has been found that the composition described in the text has a couple of advantageous properties.

The composition described in the present text can be used as dental barrier material that can be applied to gingiva or tissue in the mouth of a patient adjacent to the working area of the dentist.

Due to its hydrophilic properties, the composition sticks good to the surfaces to which it has been applied to (e.g. wet tissue).

After the composition is cured it forms an elastic film on the area to be covered. When no longer needed, the cured composition can be removed in one piece. On the contrary, removing an uncured sticky composition is more difficult. The hardness of the cured composition is also in an acceptable range.

Further, the composition is able to absorb water or other liquid or moisture (e.g. saliva or blood) in the working area in the mouth of a patient and thus helps to keep the working area dry.

The liquid-uptaking property of the composition can prevent liquids which are present in the mouth of a patient from flowing above or below the cured composition (barrier material or film) to the working field by binding the liquid to the dental barrier material.

The liquid-uptaking properties are basically twofold: In particular, due to the presence of a guanidyl containing polymer the composition is able to absorb water in its uncured stage. However, the composition is also able to bind or absorb small amounts of water on its surface in its cured stage, especially in the oxygen inhibition layer which typically occurs once the composition is cured.

Compared to known liquid rubber dam materials, the composition described in the present text is beneficial as it is able to dry the working area and keep it dry as long as the material stays in place.

Curing the barrier composition stepwise offers further advantages and may help to seal harmed tissue and absorb moisture even more effectively.

A challenge in the treatment procedure (clinical situation) is to apply a barrier material ideally exactly below the preparation line or margin of the tooth to be restored. Other areas such as those onto which dental adhesives or dental cements are to be applied during the treatment procedure later must remain freely accessibly and must not be covered with a barrier material.

If the composition described in the present text is cured stepwise, the composition comprises two sections or layers, a cured upper layer or section and an uncured or only partially cured lower layer or section.

Cured sections or layers of the composition are not sticky any longer and can thus be touched with an instrument more easily.

The cured portion or upper layer of the composition is strong enough and does not easily rupture if touched with an instrument, whereas the uncured section or lower layer of the composition remains its pasty consistency and moisture uptaking properties and is more flexible so that it can be moved or shifted on the tissue to another area.

The composition and processes described in the present text may be equally used in other treatment procedures, e.g. during an extraction of a dental alveolus, or covering any gum lesion resulting e.g. from an iatrogenic injury.

The invention relates to a composition for isolating or protecting tissue, in particular tissue in the mouth of a patient. Such a composition can also be regarded as a barrier material or composition. The composition comprises one or more radiation curable components. At least one of the radiation curable components should be a liquid at ambient conditions (e.g. 23° C.).

The composition comprises a radiation curable component with a high molecular weight polyether polyol spacer group. This radiation curable component is referred to as radiation curable component A1.

The radiation curable component A1 can be characterized by the following features alone or in combination:
a) comprising at least 2 or 3 or 4 (meth)acrylate moieties:
b) molecular weight (Mw) of polyalkylene oxide backbone: 1,000 to 20,000 g/mol: or 2,000 to 15,000 g/mol: or 3,000 to 10,000 g/mol: or 4,000 to 10,000 g/mol: or 5,000 to 10,000 g/mol: or 6,000 to 10,000 g/mol:
c) comprising a polyether polyol backbone to which (meth)acrylate moieties are attached:
d) viscosity: from 0.1 to 100 Pa*s or 1 to 50 Pa*s at 23° C.:
e) being hydrophilic (e.g. having a water contact angle <80° or <70° or <60°).

A combination of the features a) and b); or a), b) and d): or a), b) and e); or a), b), d) and e) can sometimes be preferred.

Suitable is also a radiation curable composition Al with the following features: comprising at least 2 (meth)acrylate moieties: molecular weight (Mw) of polyalkylene oxide backbone being in a range of 2,000 to 15,000 g/mol; and having a water contact angle <60°.

According to one embodiment the radiation curable component A1 comprises a polyether polyol backbone to which two (meth)acrylate moieties are attached. Such a component is in particular suitable for producing a rubber-elastic composition.

The weight average molar weight (Mw) of the polyalkylene oxide backbone is within a range of 1,000 to 20,000 g/mol, or 2,000 to 15,000 g/mol or 3,000 to 10,000 g/mol, or 4,000 to 10,000 g/mol, 5,000 to 10,000 g/mol or 6,000 to 10,000 g/mol.

A molecular weight in this range may help to improve properties like elasticity, elongation at break, Young's moduls and/or elastic modulus.

If desired, the molecular weight of the polyether polyol can be determined by titration of the OH number of the starting OH-terminated polyether component according to DIN EN ISO 4692-2. The molar weight of the radiation curable component can then be calculated by mathematically adding the molecular weight of the molecules used for chain extension and/or to introduce the (meth)acrylate functionality.

Using a hydrophilic radiation curable component in combination with the guanidyl containing polymer and the carrageenan can be advantageous as it contributes to the water-absorbing properties of the composition.

If desired, the respective properties can be determined as described in the example section.

Preferred representatives of the radiation curable components include

R—[(CH₂)ₙ—(CHR′)—O]ₖ—[(CH₂)ₘ—(CHR″)—O]ₗ—(CH₂)ₘ—(CHR″)—R
with:
n=1 to 6, preferably 1 to 4, in particular 1,
m=1 to 6, preferably 1 to 4, in particular 3,
k, l=2 to 500, preferably 4 to 250, in particular 10 to 200,
R′, R″=independently selected from H, methyl, ethyl, preferably R′=R″═H,
R═CH₂═CH—C(O)—O— or CH₂═C(CH₃)—C(O)—O—,
the bracketed expressions indexed by the symbols k and I being able to be arranged regularly or irregularly alternating or in block form.

Appropriate polyethers or polyether groups which may form the polyalkylene oxide backbone can be produced in a manner known to the person skilled in the art e.g. by the reaction of a starting compound having a reactive hydrogen atom with alkylene oxides, for example ethylene oxide, propylene oxide, butylene oxide, styrene oxide, tetrahydrofurane or epichlorohydrine or mixtures of two or more thereof.

Especially suitable are polyether compounds which are obtainable by polyaddition of ethylene oxide, 1,2-propylene oxide, 1,2-butylene oxide or tetrahydrofuran or of mixtures of two or more of the mentioned compounds with the aid of a suitable starting compound and a suitable catalyst.

The reaction products of low-molecular-weight polyfunctional alcohols having at least two hydroxyl groups with alkylene oxides, so-called polyethers, may also be used as polyols. The alkylene oxides preferably have from 2 to 4 carbon atoms. Suitable polyols are, for example, the reaction products of ethylene glycol, propylene glycol, butanediol or hexanediol isomers with one or more of the following alkylene oxides: ethylene oxide, propylene oxide or butylene oxides like tetrahydrofuran. Furthermore, the reaction products of polyfunctional alcohols such as glycerol, trimethylolethane or trimethylolpropane, pentaerythritol or sugar alcohols, or mixtures of two or more thereof, with the mentioned alkylene oxides, forming polyether polyols are also suitable.

Suitable starting compounds are, for example, water, ethylene glycol, 1,2- or 1,3-propylene glycol, 1,4- or 1,3-butylene glycol, 1,6-hexanediol, 1,8-octanediol, neopentyl glycol, 1,4-hydroxymethylcyclohexane, 2-methyl-1,3-propanediol, glycerol, trimethylolpropane, 1,2,6-hexanetriol, 1,2,4-butanetriol, trimethylolethane, pentaerythritol, mannitol, sorbitol, or mixtures of two or more thereof.

Especially suitable are polyether compounds as are obtainable by polyaddition of ethylene oxide, 1,2-propylene oxide, 1,2-butylene oxide or tetrahydrofuran or of mixtures of two or more of the mentioned compounds with the aid of a suitable starting compound and a suitable catalyst.

For example, polyether polyols which are prepared by copolymerisation of tetrahydrofuran and ethylene oxide in a molar ratio of 10:1 to 1:1, preferably 10:1 to 4:1, in the presence of strong acids, for example boron fluoride etherates, are suitable as well.

Specific examples of the radiation curable component include (meth)acrylated ethylene oxide, propylene oxide, ethylene/propylene oxide copolymers, ethylene oxide/tetrahydrofuran copolymers, polypropylene glycol and mixtures thereof.

The radiation curable component A1 is typically present in the following amounts: lower amount: at least 40 or at least 45 or at least 50 wt. %: upper amount: at most 90 or at most 85 or at most 80 wt. %: range: 40 to 90 or 45 to 85 or 50 to 80 wt. %: wt. % with respect to the whole composition.

The curable composition may also comprise in addition a urethane methacrylate, which is different from the radiation curable component comprising the polyether polyol spacer group and the at least two (meth)acrylate moieties.

Adding a (meth)acrylate components with a urethane moiety may help to improve physical properties of the cured composition like flexural strength and/or elongation at break.

The urethane methacrylate may be characterized by the following features alone or in combination:
a) comprising at least 2 or 3 or 4 (meth)acrylate moieties;
b) molecular weight (Mw): 200 to 1,000 g/mol or 300 to 800 g/mol;
c) comprising a C₁ to C₂₀ linear or branched alkyl moiety to which the (meth)acrylate moieties are attached through the urethane moieties;
d) viscosity: from 0.1 to 100 Pa*s or 1 to 50 Pa*s at 23° C.

A combination of the features a) and b) or b) and c) or a) and d) can sometimes be preferred.

Suitable examples of urethane (meth)acrylates include 7,7,9-trimethyl-4,13-dioxo-3,14-dioxa-5,12-diazahexadecane-1,16-dioxy-dimethacrylate (e.g. Visiomer™ HEMATMDI, Evonik), 7,7,9-trimethyl-4,13-dioxo-5,12-diazahexadecane-1,16-dioxy-dimethacrylate (UDMA), urethane (methacrylates) derived from 1,4 and 1,3-Bis(1-isocyanato-1-methylethyl)bezene (e.g. as described in EP 0 934 926 A1) and mixtures thereof.

A suitable urethane dimethacrylate can be characterized e.g. by the following formula:

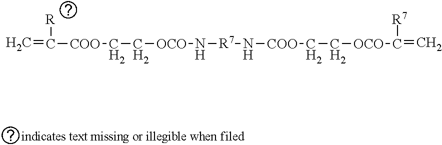

wherein R¹ is a hydrogen atom or a methyl group, and R² is a linear or branched alkylene group of 1 to 8 carbon atoms or

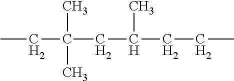

Specific examples of urethane (meth)acrylate also include di(acryloxyethyl)dimethylene diurethane, di(methacryloxyethyl)-dimethylene diurethane, di(acryloxyethyl)tetramethylene diurethane, di(methacryloxyethyl)-tetramethylene diurethane, di(acryloxyethyl)-trimethyl-hexamethylene diurethane, and di(methcryloxyethyl)-trimethylhexanmethylene diurethane, and mixtures thereof.

The urethane methacrylate may be present in the following amounts: lower amount: 0 or at least 1 or at least 5 wt. %; upper amount: at most 40 or at most 35 or at most 30 wt. %; range: 0 to 40 or 1 to 35 or 5 to 30 wt. %; wt. % with respect to the whole composition.

Besides the high molecular weight radiation curable component A1 described above, the composition may comprise further lower molecular weight radiation curable components A2 not comprising a urethane moiety. The radiation curable components A2 typically have a molecular weight in a range of 130 to 800 g/mol.

Suitable components include hydrophilic monomers such as 2-hydroxylethylmethacrylate or liquid acrylate or methacrylate functionalized homo- or copolymers of ethylene glycol, propylene glycol and THF. Some of the radiation curable components A2 may be characterized by the following formula

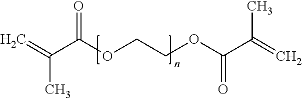

with n=4 to 14

More specific examples include polyethylene glycol dimethacrylate having a molecular weight (Mw) in the range of 330 to 750 g/mol.

If present, the radiation curable component A2 is typically present in an amount lower than the amount of the radiation curable component A1.

The radiation curable component A2 may be present in the following amounts: lower amount: 0 or at least 1 or at least 5 wt. %; upper amount: at most 30 or at most 25 or at most 20 wt. %; range: 0 to 30 or 1 to 25 or 5 to 20 wt. %; wt. % with respect to the whole composition.

Thus, the composition may comprise a radiation curable component A1 as described in the present text and a radiation curable component A2 having a molecular weight in the range of 130 to 800 g/mol and being present in an amount lower than the amount of radiation curable component A1.

The polymerizable composition comprises one or more photo-initiators.

The nature and structure of the photo-initiator is not particularly limited unless the intended purpose cannot be achieved. Suitable photo initiator(s) for free radical polymerization are generally known to the person skilled in the art dealing with dental materials.

As photo-initiator(s), those which can polymerize the radiation curable components by the action of visible light having a wavelength in the range of 430 nm to 700 nm are preferred.

Suitable photo-initiator(s) often contain an alpha di-keto moiety, an anthraquinone moiety, a thioxanthone moiety or benzoin moiety.

Examples of photo-initiator(s) include camphor quinone, 1-phenyl propane-1,2-dione, benzil, diacetyl, benzyl dimethyl ketal, benzyl diethyl ketal, benzyl di (2-methoxyethyl) ketal, 4,4,′-di-methylbenzyl dimethyl ketal, anthraquinone, 1-chloroanthraquinone, 2-chloroanthraquinone, 1,2-benzanthraquinone, 1-hydroxyanthraquinone, 1-methylanthraquinone, 2-ethylanthraquinone, 1-bromoanthraquinone, thioxanthone, 2-isopropyl thioxanthone, 2-nitrothioxanthone, 2-methyl thioxanthone, 2,4-dimethyl thioxanthone, 2,4-diethyl thioxanthone, 2,4-diisopropyl thioxanthone, 2-chloro-7-trifluoromethyl thioxanthone, thioxanthone-10,10-dioxide, thioxanthone-10-oxide, benzoin methyl ether, benzoin ethyl ether, isopropyl ether, benzoin isobutyl ether, benzophenone, bis(4-dimethylaminophenyl)ketone, 4,4,′-bisdiethylaminobenzophenone.

Using acylphosphine oxides was found to be useful, as well. Using acylphosphine oxides can sometimes be preferred. Suitable acylphosphine oxides can be characterized by the following formula

(R⁹)₂¾P(=O)¾C(=O)—R¹⁰
wherein each R⁹ individually can be a hydrocarbyl group such as alkyl, cycloalkyl, aryl, and aralkyl, any of which can be substituted with a halo-, alkyl- or alkoxy-group, or the two R⁹ groups can be joined to form a ring along with the phosphorous atom, and wherein R¹⁰ is a hydrocarbyl group, an S—, O—, or N-containing five- or six-membered heterocyclic group, or a —Z—C(═O)—P(═O)—(R⁹)₂ group, wherein Z represents a divalent hydrocarbyl group such as alkylene or phenylene having 2 to 6 carbon atoms.

Suitable systems are also described e.g. in U.S. Pat. No. 4,737,593 (Ellrich et al.), the content of which is herewith incorporated by reference.

Preferred acylphosphine oxides are those in which the R⁹ and R¹⁰ groups are phenyl or lower alkyl- or lower alkoxy-substituted phenyl. By “lower alkyl” and “lower alkoxy” is meant such groups having from 1 to 4 carbon atoms. In particular, 2,4,6-trimethylbenzoyl diphenyl phosphine oxide was found to be useful (Lucirin™ TPO, BASF).

More specific examples include: bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenyl-phosphine oxide, bis-(2,6-dichlorobenzoyl)-4-biphenylylphosphine oxide, bis-(2,6-dichloro-benzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chloro-phenylphosphine oxide, bis-(2,6-dichlorobenzoyl)-2,4-dimethoxyphenylphosphine oxide, bis-(2,6-dichlorobenzoyl) decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxybenzoyl)-phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-3,4,5-trimethoxybenzoyl)-4-ethoxyphenylphosphine oxide, bis-(2-methyl-1-naphthoyl)-2,5-dimethyl-phenylphosphine oxide, bis-(2-methyl-1-naphthoyl)phenylphosphine oxide, bis-(2-methyl-1-naphthoyl)-4-biphenylylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide and bis-(2-chloro-1-naphthoyl)-2,5-dimethylphenylphosphine oxide.

The acylphosphine oxide bis(2,4,6-trimethylbenzoyl)phenyl phosphine oxide (contained in Omnirad™ 2022; iGM Resins) is sometimes preferred.

Besides the photo-initiator, a reducing agent might be present. The combination of a photo-initiator and a reducing agent is often referred to as photo-initiator system.

As reducing agent or donor component, tertiary amines are generally used.

Suitable examples of the tertiary amines include N,N-dimethyl-p-toluidine, N,N-dimethyl-aminoethyl methacrylate, triethanolamine, methyl 4-dimethylaminobenzoate, ethyl 4-dimethyl-aminobenzoate, methyldiphenylamine and isoamyl 4-dimethylaminobenzoate.

The photo-initiator is typically present in the following amounts: lower amount: at least 0.1 or at least 0.2 or at least 0.3 wt. %; upper amount: at most 5 or at most 4 or at most 3 wt. %; range: 0.1 to 5 or 0.2 to 4 or 0.3 to 3 wt. %; wt. % with respect to the whole composition.

The polymerizable composition comprises a guanidinyl-containing polymer as water- or liquid absorbing component.

Compared to other known water-absorbing components such as so-called superabsorbers, which are particulate salts of crosslinked polyacrylic acids, using a guanidinyl-containing polymer may help to reduce the extrusion force and/or flow resistance of the composition.

The term “guanidinyl-containing polymer” includes also polymers where the guanidinyl moiety is present in its protonated form including the salts thereof (in particular chloride and sulphate salts).

Suitable polymers include polyvinylamine, poly(N-methylvinylamine), polyallylamine, poly-allylmethylamine, polydiallylamine, poly(4-aminomethylstyrene), poly(4-aminostyrene), poly-(acrylamide-co-methylaminopropylacrylamide), poly(acrylamide-co-aminoethylmethacrylate), polyethylenimine, polypropylenimine, polylysine, polyaminoamides, polydimethylamine-epichlorohydrin-ethylenediamine, polyaminosiloxanes, dendrimers formed from polyamidoamine and polypropylenimine, biopolymers, polyacrylamide homo- or copolymers, amino-containing polyacrylate homo- or copolymers.

For some embodiments, the preferred amino-containing polymers include polyaminoamides, polyethyleneimine, polyvinylamine, polyallylamine, polydiallylamine and acrylamide-based polymers.

As used herein, the term “guanidinyl” refers to a group of the following formula

—NR³—C(=NR⁴)—NR⁴R⁵.

If the guanidinyl group is part of a pendant group, the group R³ refers to hydrogen, C₁-C₁₂ (hetero)alkyl, or C₅-C₁₂ (hetero)aryl.

If the guanidinyl group is part of the backbone of the polymer, the group R³ can refer to a residue of a polymer chain.

Each group R⁴ is independently hydrogen, C₁-C₁₂ (hetero)alkyl, or C₅-C₁₂ (hetero)aryl. Group R⁵ is hydrogen, C₁-C₁₂ (hetero)alkyl, C₅-C₁₂ (hetero)aryl, or a group of formula —N(R⁴)₂.

The guanidinyl group can be part of a biguanidinyl group that is of formula —NR³—C(═NR⁴)—NR⁴—C(═NR⁴)—NR⁴R⁵ where the groups R³, R⁴, and R⁵ are the same as defined above.

Although any guanidinyl-containing polymer can be used in the cationic form, this polymer is often of Formula (I).

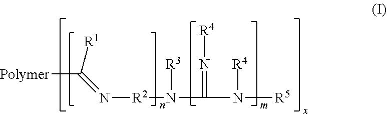

In Formula (I), the group R¹ is hydrogen, C₁-C₁₂ (hetero)alkyl, or C₅-C₁₂ (hetero)aryl, or a residue of the polymer chain. The group R² is a covalent bond, a C₂-C₁₂ (hetero)alkylene, or a C₅-C₁₂ (hetero)arylene. The group R³ is H, C₁-C₁₂ (hetero)alkyl, or C₅-C₁₂ (hetero)aryl, or can be a residue of the polymer chain when n is 0. Each group R⁴ is independently hydrogen, C₁-C₁₂ (hetero)alkyl, or C₅-C₁₂ (hetero)aryl. The group R⁵ is hydrogen, C₁-C₁₂ (hetero)alkyl, C₅-C₁₂ (hetero)aryl, or —N(R⁴)₂. The variable n is equal to 0 or 1 depending on the precursor polymer used to form the guanidinyl-containing polymer. The variable m is equal to 1 or 2 depending on whether the cationic group is a guanidinyl or biguanidinyl group. The term “Polymer” in Formula (I) refers to all portions of the guanidinyl-containing polymer except the x groups of formula —[C(R¹)═N—R²—]ₙN(R³)—[C(═NR⁴)—NR⁴R⁵—]ₘ. The term x is a variable equal to at least 1.

Most guanidinyl-containing polymers have more than one guanidinyl group. The number of guanidinyl groups can be varied depending the method used to prepare the guanidinyl-containing polymer. For example, the number of guanidinyl groups can depend on the choice of precursor polymer selected for reacting with a suitable guanylating agent. In some embodiments, the variable x can be up to 1000, up to 500, up to 100, up to 80, up to 60, up to 40, up to 20, or up to 10.

The guanidinyl-containing polymer of Formula (I) is often the reaction product of (a) a precursor polymer and (b) a suitable guanylating agent.

The precursor polymer is often an amino-containing polymer or a carbonyl-containing polymer. When the precursor polymer is an amino-containing polymer, the variable n in Formula (I) is typically equal to 0. When the precursor polymer is a carbonyl-containing polymer, the variable n is equal to 1. If the guanylating agent contains a guanidinyl group or a precursor of a guanidinyl group, the variable m in Formula (I) is equal to 1. If the guanylating agent contains a biguanidinyl group or a precursor of a biguanidinyl group, the variable m in Formula (I) is equal to 2.

In embodiments where n is 0, the base polymer of the guanidinyl-containing polymer is often prepared by reaction of a suitable guanylating agent and an amino-containing polymer. In other embodiments, where n is 1, the guanidinyl-containing polymer is often prepared by reaction of a suitable guanylating agent and a carbonyl-containing polymer.

In those embodiments where n is 0 and the precursor polymer is an amino-containing polymer, the structure of the guanidinyl-containing polymer of Formula (I) can also be written more simply as the structure of Formula (II).

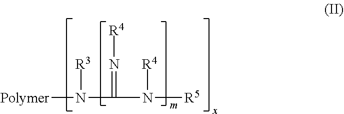

In Formula (II), the group R³ is hydrogen, C₁-C₁₂ (hetero)alkyl, or C₅-C₁₂ (hetero)aryl, or can be a residue of the polymer chain. When the guanidinyl group is part of a pendant group, R³ is hydrogen, C₁-C₁₂ (hetero)alkyl, or C₅-C₁₂ (hetero)aryl. Each R⁴ is independently hydrogen, C₁-C₁₂ (hetero)alkyl, or C₅-C₁₂ (hetero)aryl. The group R⁵ is hydrogen, C₁-C₁₂ (hetero)alkyl, or C₅-C₁₂ (hetero)aryl, or —N(R⁴)₂. The variable m is equal to 1 or 2. The term “Polymer” in the formula above refers to all portions of the guanidinyl-containing polymer except the x groups of formula —N(R³)—[C(═NR⁴)—NR⁴R⁵—]ₘ. The term x is a variable equal to at least 1.

The amino-containing polymer used as a precursor polymer to prepare a guanidinyl-containing polymer of Formula (II) can be represented by the formula Polymer —N(R³)H. As noted above, however, the amino-containing polymer typically has many groups —N(R³)H but Formula (I) shows only one for ease of discussion purposes only. The —N(R³)H groups can be a primary or secondary amino group and can be part of a pendant group or part of the backbone of the precursor polymer.

The amino-containing polymers can be synthesized or can be naturally occurring biopolymers. Suitable amino-containing polymers can be prepared by chain growth or step growth polymerization procedures with amino-containing monomers. These monomers can also, if desired, be copolymerized with other monomers without an amino-containing group. Additionally, the amino-containing polymers can be obtained by grafting primary or secondary amine groups using an appropriate grafting technique.

The guanidinyl-containing polymer also includes polymers where the guanidinyl moiety is protonated including polymers having the following formula:

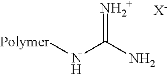

with X⁻ being selected from Cl⁻, Br⁻, I⁻, ½SO₄²⁻, NO₃⁻, CH₃COO⁻, C₃H₇COO⁻.

Examples of amino-containing polymers suitable for use, which are prepared by chain growth polymerization include, but are not limited to, polyvinylamine, poly(N-methylvinylamine), polyallylamine, polyallylmethylamine, polydiallylamine, poly(4-aminomethylstyrene), poly(4-aminostyrene), poly(acrylamide-co-methylaminopropylacrylamide), and poly(acrylamide-co-aminoethylmethacrylate).

Examples of amino-containing polymers suitable for use, which are prepared by step growth polymerization include, but are not limited to, polyethylenimine, polypropylenimine, polylysine, polyaminoamides, polydimethylamine-epichlorohydrin-ethylenediamine, and any of a number of polyaminosiloxanes, which can be prepared from monomers such as aminopropyltriethoxysilane, N-(2-aminoethyl)-3-aminopropyltrimethoxysilane, N-trimethoxysilylpropyl-N-methylamine, and bis(trimethoxysilylpropyl) amine.

Other useful amino-containing polymers that have primary or secondary amino end groups include, but are not limited to, dendrimers (hyperbranched polymers) formed from polyamidoamine (PAMAM) and polypropylenimine. Exemplary dendrimeric materials formed from PAMAM are commercially available under the trade designation “STARBURST (PAMAM) dendrimer” (e.g., Generation 0 with 4 primary amino groups, Generation 1 with 8 primary amino groups, Generation 2 with 16 primary amino groups, Generation 3 with 32 primary amino groups, and Generation 4 with 64 primary amino groups) from Aldrich Chemical (Milwaukee, WI). Dendrimeric materials formed from polypropylenimine are commercially available under the trade designation “DAB-Am” from Aldrich Chemical. For example, DAB-Am-4 is a generation 1 polypropylenimine tetraamine dendrimer with 4 primary amino groups, DAB-Am-8 is a generation 2 polypropylenimine octaamine dendrimer with 8 primary amino groups, DAB-Am-16 is a generation 3 polypropylenimine hexadecaamine with 16 primary amino groups, DAB-Am-32 is a generation 4 polypropylenimine dotriacontaamine dendrimer with 32 primary amino groups, and DAB-Am-64 is a generation 5 polypropylenimine tetrahexacontaamine dendrimer with 64 primary amino groups.

Examples of suitable amino-containing polymers that are biopolymers include chitosan as well as starch that is grafted with reagents such as methylaminoethylchloride.

Still other examples of amino-containing polymers include polyacrylamide homo- or copolymers and amino-containing polyacrylate homo- or copolymers prepared with a monomer composition containing an amino-containing monomer such as an aminoalkyl (meth)acrylate, (meth)acrylamido-alkylamine, and diallylamine.

For some embodiments, the preferred amino-containing polymers include polyaminoamides, polyethyleneimine, polyvinylamine, polyallylamine, and polydiallylamine.

Suitable commercially available amino-containing polymers include, but are not limited to, polyamidoamines that are available under the trade designations ANQUAMINE™ (e.g., ANQUAMINE™ 360, 401, 419, 456, and 701) from Air Products and Chemicals (Allentown, PA), polyethylenimine polymers that are available under the trade designation LUPASOL™ (e.g., LUPASOL™ FG, PR 8515, Waterfree, P, and PS) from BASF Corporation (Resselaer, NY), polyethylenimine polymers such as those available under the trade designation CORCATT P-600 from EIT Company (Lake Wylie, SC), and polyamide resins such as those available from Cognis Corporation (Cincinnati, OH) under the traded designation VERSAMID™ series of resins that are formed by reacting a dimerized unsaturated fatty acid with alkylene polyamines.

Guanidinyl-containing polymers can be prepared by reaction of the amino-containing polymer precursor with a guanylating agent.

Although all the amino groups of the amino-containing polymer can be reacted with the guanylating agent, there are often some unreacted amino groups from the amino-containing polymer precursor remaining in the guanidinyl-containing polymer. Typically, at least 0.1 mole percent, at least 0.5 mole percent, at least 1 mole percent, at least 2 mole percent, at least 10 mole percent, at least 20 mole percent, or at least 50 mole percent of the amino groups in the amino-containing polymer precursor are reacted with the guanylating agent. Up to 100 mole percent, up to 90 mole percent, up to 80 mole percent, or up to 60 mole percent of the amino groups can be reacted with the guanylating agent. For example, the guanylating agent can be used in amounts sufficient to functionalize 0.1 to 100 mole percent, 0.5 to 90 mole percent, 1 to 90 mole percent, 1 to 80 mole percent, 1 to 60 mole percent, 2 to 50 mole percent, 2 to 25 mole percent, or 2 to 10 mole percent of the amino groups in the amino-containing polymer.

Known guanylating agents for reaction with an amino-containing polymer precursor include, but are not limited to, cyanamide: O-alkylisourea salts such as O-methylisourea sulfate, O-methylisourea hydrogen sulfate, O-methylisourea acetate, O-ethylisourea hydrogen sulfate, and O-ethylisourea hydrochloride: chloroformamidine hydrochloride: 1-amidino-1,2,4-triazole hydro-chloride: 3,5-dimethylpyrazole-1-carboxamidine nitrate: pyrazole-1-carboxamidine hydrochloride: N-amidinopyrazole-1-carboxamidine hydrochloride; and carbodiimides such as dicyclohexyl-carbodiimide, N-ethyl-N′-(3-dimethylaminopropyl) carbodiimide, and diisopropylcarbodiimide. The amino-containing polymer may also be acylated with guanidino-functional carboxylic acids such as guanidinoacetic acid and 4-guanidinobutyric acid in the presence of activating agents such as EDC (N-[3-(dimethylamino)propyl]-3-ethylcarbodiimide hydrochloride), or EEDQ (2-ethoxy-1-ethoxycarbonyl-1,2-dihydroquinoline). Additionally, the guanidinyl-containing polymer may be prepared by alkylation with chloroacetone guanyl hydrazone, e.g. as described in U.S. Pat. No. 5,712,027 (Ali et al.).

Guanylating agents for the preparation of biguanide-containing polymers include sodium dicyanamide, dicyanodiamide and substituted cyanoguanidines such as N³-p-chlorophenyl-N¹-cyanoguanidine, N³-phenyl-N¹-cyanoguanidine, N³-alpha-naphthyl-N¹-cyanoguanidine, N³-methyl-N¹-cyanoguanidine, N³,N³-dimethyl-N¹-cyanoguanidine, N³-(2-hydroxyethyl)-N¹-cyanoguanidine, and N³-butyl-N¹-cyanoguanidine. Alkylene- and arylenebiscyanoguanidines may be utilized to prepare biguanide functional polymers by chain extension reactions. The preparation of cyanoguanidines and biscyanoguanidines is described in detail in Rose, F. L. and Swain, G. J. Chem Soc., 1956, pp. 4422-4425. Other useful guanylating reagents are described e.g. by Alan R. Katritzky et al., Comprehensive Organic Functional Group Transformation, Vol. 6, p. 640.

The guanidinyl-containing polymer formed by reaction of an amino-containing polymer precursor and a guanylating agent will have pendent or catenary guanidinyl groups of the Formula (III).

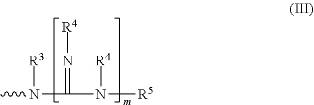

In Formula (III), the groups R³, R⁴, and R⁵ and the variable m are the same as defined above. The wavy line attached to the N(R³) group shows the position of attachment the group to the rest of the polymeric material. In most embodiments, the group of Formula (III) is in a pendant group of the guanidinyl-containing polymer.

In some embodiments, it may be advantageous to react the amino-containing polymer precursor to provide other ligands or groups in addition to the guanidinyl-containing group. For example, it may be useful to include a hydrophobic ligand, an ionic ligand, or a hydrogen bonding ligand. This can be particularly advantageous for the removal of certain microorganisms during the wiping of a microorganism-contaminated surface.

The additional ligands can be readily incorporated into the amino-containing polymers by alkylation or acylation procedures well known in the art. For example, amino groups of the amino-containing polymer can be reacted using halide, sulfonate, and sulfate displacement reactions or using epoxide ring opening reactions. Useful alkylating agents for these reactions include, for example, dimethylsulfate, butyl bromide, butyl chloride, benzyl bromide, dodecyl bromide, 2-chloroethanol, bromoacetic acid, 2-chloroethyltrimethylammonium chloride, styrene oxide, glycidyl hexadecyl ether, glycidyltrimethylammonium chloride, and glycidyl phenyl ether. Useful acylating agents include, for example, acid chlorides and anhydrides such as benzoyl chloride, acetic anhydride, succinic anhydride, and decanoyl chloride, and isocyanates such as trimethylsilylisocyanate, phenyl isocyanate, butyl isocyanate, and butyl isothiocyanate. In such embodiments 0.1 to 20 mole percent, preferably 2 to 10 mole percent, of the available amino groups of the amino-containing polymer may be alkylated and/or acylated.

The guanidinyl-containing polymer can be crosslinked. The amino-containing polymer can be crosslinked prior to reaction with the guanylating agent. Alternatively, the guanidinyl-containing polymer can be crosslinked by reaction of a crosslinker with remaining amino groups from the amino-containing polymer precursor or with some of the guanidinyl groups. Suitable crosslinkers include amine-reactive compounds such as bis- and polyaldehydes such as glutaraldehyde, bis- and polygylcidylethers such as butanedioldiglycidylether and ethyleneglycoldiglycidylether, polycarboxylic acids and their derivatives (e.g., acid chlorides), polyisocyanates, formaldehyde-based crosslinkers such as hydroxymethyl and alkoxymethyl functional crosslinkers, such as those derived from urea or melamine, and amine-reactive silanes, such as 3-glycidoxypropyltrimethoxysilane, 3-glycidoxypropyltriethoxysilane, 5,6-glycidoxypropyltrimethoxysilane, epoxyhexyltriethoxysilane, (p-chloromethyl)phenyltrimethoxysilane, chloromethyltriethoxysilane, 3-isocyanatopropyltriethoxysilane, and 3-thiocyanatopropyltriethoxysilane.

In other embodiments, the guanidinyl-containing polymer is of Formula (IV), which corresponds to Formula (I) where n is equal to 1.

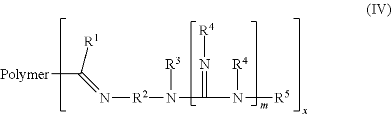

In Formula (IV), the group R¹ is hydrogen, C₁-C₁₂ (hetero)alkyl, or C₅-C₁₂ (hetero)aryl, or a residue of the polymer chain. If the guanidinyl-containing group is the reaction product of a guanylating agent and a carbonyl group that is part of the backbone of the polymer, R¹ is a residue of the polymer chain. Group R² is a covalent bond, a C₂-C₁₂ (hetero)alkylene, or a C₅-C₁₂ (hetero)arylene. Group R³ is hydrogen, C₁-C₁₂ (hetero)alkyl, or C₅-C₁₂ (hetero)aryl. Each R⁴ is independently H, C₁-C₁₂ (hetero)alkyl, or C₅-C₁₂ (hetero)aryl. Group R⁵ is hydrogen, C₁-C₁₂ (hetero)alkyl, or C₅-C₁₂ (hetero)aryl, or —N(R⁴)₂. The variable m is equal to 1 or 2. The term “Polymer” in Formula (I) refers to all portions of the guanidinyl-containing polymer except the x groups of formula —C(R¹)═N—R²—N(R³)—[C(═NR⁴)—NR⁴R⁵—]ₘ. The term x is a variable equal to at least 1.

Guanidinyl-containing polymers of Formula (IV) are the reaction product of a carbonyl-containing polymer and a suitable guanylating agent for reaction with a carbonyl group. The carbonyl-containing polymer used as a precursor polymer to prepare a guanidinyl-containing polymer of Formula (IV) can be represented by the formula Polymer —C(O)—R¹. The carbonyl-containing polymer precursor typically has many groups —C(O)—R¹ but Formula (IV) shows only one for ease of discussion purposes only. The carbonyl group —C(O)—R¹ is an aldehyde group (when RI is hydrogen) or a ketone groups (when R¹ ia a (hetero)alkyl or (hetero)aryl). Although the carbonyl-group can be part of the polymeric backbone or part of a pendant group from the polymeric backbone, it is typically in a pendant group.

If desired, the guanidinyl-containing polymers can be produced as described in US 2016/0115430 A1 (Swanson et al.), in particular in sections [0049] to [0080], the description of which is herewith incorporated by reference.

The guanidyl containing polymer is typically present in the following amounts: lower amount: at least 2 or at least 3 or at least 4 wt. %: upper amount: at most 10 or at most 9 or at most 8 wt. %: range: 2 to 10 or 3 to 9 or 4 to 8 wt. %; wt. % with respect to the whole composition.

The curable composition may also comprise one or more carageenans.

Carrageenans or carrageenins are a family of sulphated polysaccharides that are typically extracted from red edible seaweeds. There are three main varieties of carrageenan, which differ in their degree of sulphation.

Kappa-carrageenan has one sulphate group per disaccharide. Iota-carrageenan has two sulphates per disaccharide. Lambda carrageenan has three sulphates per disaccharide. Other carrageenan(s) which are known are epsilon and u.

With respect to the present text, the use of iota or lambda carrageenan(s) can sometimes be preferred.

Carrageenans are large, highly flexible molecules that curl forming helical structures. This gives them the ability to form a variety of different gels at room temperature.

Carrageenans are polysaccharides made up of repeating galactose units and 3,6 anhydrogalactose (3,6-AG), both sulfated and non-sulfated. The units are typically joined by alternating α-1,3 and β-1,4 glycosidic linkages.

If desired, the carrageenan(s) can be characterized by the following features alone or in combination: molecular weight (Mw): 10,000 to 1,000,000 or 20,000 to 500,000 g/mol: ester sulphate content: 25 to 40 wt. % or 25 to 30 wt. % with respect to the weight of the carrageenan.

The carrageenan(s) may be present in the following amounts: lower amount: at least 2 or at least 3 or at least 4 wt. %: upper amount: at most 10 or at most 9 or at most 8 wt. %: range: 2 to 10 or 3 to 9 or 4 to 8 wt. %: wt. % with respect to the composition.

If present, the ratio of guanidinyl-containing polymer(s) to carrageenan(s) is typically in a range of 2:1 to 1:2 with respect to weight.

According to one embodiment, the guanidinyl-containing polymer and the carrageenan component are used in essentially equal amounts with respect to weight.

A suitable composition may comprise the guanidinyl-containing polymer in an amount of 2 to 8 wt. % and the carrageenan component in an amount of 2 to 8 wt. %.

The ratio of radiation curable components to water-absorbing components is typically in a range of 4:1 to 2:1 with respect to weight. Such a range was found to be beneficial as it provides a good balance between liquid-absorbing properties, swelling behaviour and mechanical properties of the composition after hardening.

The polymerizable composition may also comprise a softener.

Suitable softeners can typically be characterized by the following features alone or in combination: being non-aqueous: being hydrophilic: having a boiling point above 100° C.; having a molecular weight (Mw) of less than 1,000 g/mol, preferably in a range of 60 to 1,000 g/mol, or 100 to 800 g/mol.

Softeners which can be used include glycol, glycerine, ethylene glycol, poly(ethylene glycol), propylene glycol, poly(propylene glycol), and mixtures thereof. The use of poly(ethylene glycol) or poly(propylene glycol) is sometimes preferred.

With increasing amount of the softener, the viscosity and surface hardness of set material of the composition can be adjusted, in particular lowered. Good paste properties can be obtained, if the softener is used in the amounts given below.

Softener may be present in the following amounts: lower amount: 0 or at least 0.5 or at least 1 wt. %: upper amount: at most 20 or at most 15 or at most 10 wt. %; range: 0 to 20 or 0.5 to 15 or 1 to 10 wt. %; wt. % with respect to the whole composition.

The polymerizable composition may further comprise one or more dyes.

The presence of a dye typically provides colour to the composition and makes it better visible to the practitioner during use. Further, as a dye absorbs radiation, it is a means for adjusting the curing behaviour and in particular the curing depth of a radiation-curable composition.

Dyes which were found to be suitable for the present invention may be characterized by the following features alone or in combination:
a) solubility: at least 0.1 g in 100 g triethylenglycole di(meth)acrylate (TEGDMA) at 23° C.:
b) having a light absorption maximum in the range of 420 to 600 nm:
c) having a fluorescence band in the range of 500 to 650 nm;
d) comprising a perylene or naphtalimide moiety.

A combination of the features a) and b) or a), b) and c) or a), b), c) and d) can sometimes be preferred.

Such a light absorption may help to adjust the curing depth of the composition more effectively, e.g. to ensure that only the upper section or layer of the composition is cured, but that the lower section or layer of the composition remains uncured or only in a partially cured stage.

Suitable examples include red dyes like Lumogen™ F Red 300 (BASF) and Fluoreszenzrot 94720 (Kremer) having an absorption maximum at about 575 nm, orange dyes like Fluoreszenzorange 94738 (Kremer) having an absorption maximum at about 526 nm and yellow dyes like Fluoreszenzgelb 94700 (Kremer) having an absorption maximum at about 474 or 476 nm or Lumugen™ F yellow 083 (BASF).

The dye may be present in the following amount: lower amount: 0 or at least 0.005 or at least 0.01 wt. %: upper amount: at most 1 or at most 0.8 or at most 0.5 wt. %; range: 0 to 1 or 0.005 to 0.8 or 0.1 to 0.5 wt. %: wt. % with respect to the whole composition.

The polymerizable composition may further comprise one or more fillers.

Fillers which can be used comprise non acid reactive fillers. A non-acid reactive filler is a filler which does not undergo an acid/base reaction with an acid.

Useful non acid reactive fillers include alumina, ground glasses, quartz, fumed silica, silica gels such as silicic acid and combinations thereof.

Suitable fumed silicas include for example, products sold under the tradename Aerosil™ series OX-50, -130, -150, and -200, Aerosil™ R8200, R805 available from Evonik, CAB-O-SIL™ M5 available from Cabot Corp (Tuscola), and HDK types e.g. HDK™-H2000, HDK™ H15, HDK™ H18, HDK™ H20 and HDK™ H30 available from Wacker.

Surface-treating of the filler particles before combining with the radiation curable component may provide a more stable dispersion in the resin. The surface-treatment may help to stabilize the filler particles so that the particles are well-dispersed and yielding a substantially homogeneous polymerizable composition.

According to one embodiment the silane treatment agents are able to polymerize with the radiation curable components such as gamma-methacryloxypropyltrimethoxysilane, available under the trade designation A-174 from Witco OSi Specialties (Danbury, Conn.).

According to another embodiment and sometimes preferred are silane treatment agents, which are not able to react with curable components. Examples of silanes of this type include, for example, alkyl, hydroxy alkyl, aryl, hydroxy aryl or amino functional silanes.

Filler may be present in the following amounts: lower amount: 0 or at least 0.1 or at least 1 wt. %: upper amount: at most 15 or at most 12 or at most 10 wt. %; range: 0 to 15 or 0.5 to 12 or 1 to 10 wt. %: wt. % with respect to the whole composition.

The polymerizable composition may further comprise one or more additives.

The composition described in the present text may contain additives such as solvents, flavouring agents, stabilizers, UV absorbers, anti-microbials, colourants and/or fragrances.

Solvents, which can be present include linear, branched or cyclic, saturated or unsaturated alcohols, ketones, esters, ethers or mixtures of two or more of said type of solvents with 2 to 10 C atoms. Preferred alcoholic solvents include methanol, ethanol, iso-propanol and n-propanol. Other suitable organic solvents are THF, acetone, methyl ethyl ketone, cyclohexanol, toluene, alkanes and acetic acid alkyl esters, in particular acetic acid ethyl ester.

Typical flavouring agent(s), which can be used, include but are not limited to isoamylacetate (banana), benzaldehyde (bitter almond). Cinnamic aldehyde (Cinnamon), ethylpropionate (fruity), methyl anthranilate (Grape), mints (e.g. peppermints), limonene (e.g. Orange), allylhexanoate (pineapple), ethylmaltol (candy), ethylvanillin (Vanilla), methylsalicylate (Wintergreen).

Stabilizer(s) which can be used often comprise a phenol moiety or phosphonic acid moieties. Specific examples of stabilizer(s) which can be used include: p-methoxyphenol (MOP), hydroquinone monomethylether (MEHQ), 2,6-di-tert-butyl-4-methyl-phenol (BHT; Ionol), phenothiazine, 2,2,6,6-tetramethyl-piperidine-1-oxyl radical (TEMPO) Vitamin E: N,N′-di-2-butyl-1,4-phenylenediamine; N,N′-di-2-butyl-1,4-phenylenediamine; 2,6-di-tert-butyl-4-methylphenol; 2,4-dimethyl-6-tert-butylphenol; 2,4-dimethyl-6-tert-butylphenol and 2,6-di-tert-butyl-4-methylphenol; 2,6-di-tert-butylphenol; pentaerythritoltetrakis(3-(3,5-di-tert-butyl-4-hydroxyphenyl)propionate) (previously known as Irganox™ 1010); octyl-3,5-di-tert-butyl-4-hydroxy-hydrocinnamate; octadecyl 3-(3,5-di-tert-butyl-4-hydroxyphenyl)propionate; 1,3,5-trimethyl-2,4,6-tris(3,5-di-tert-butyl-4-hydroxybenzyl)benzene; 2,2′,4,4′-tetrakis-tert-butyl-3,3′-dihydroxybiphenyl; 4,4-Butylidenebis (6-tert-butyl-m-cresol); 4,4′-Isopropyliden-bis-(2-tert-butylphenol): 2,2′-methylene-bis(6-nonyl-p-cresol); 1,3,5-tris(3,5-di-tert-butyl-4-hydroxybenzyl-)-1,3,5-triazine-2,4,6-(1H,3H,5H)trione; pyrogallol; N-nitroso-N-phenylhydroxylamine; 3-propenylphenol, phenothiazine, N-phenyl-2-naphthylamine, phosphorous acid phenylphosphonic acid; vinylphosphonic acid or combinations or mixtures thereof.

Adding a colorant in addition to and being different from the dye described above to the composition can be advantages as it may further facilitates the application process of the composition in the mouth of a patient and the control whether after the treatment all residues of the composition have been removed.

E.g., blue or green colours may be suitable. Examples of colourants which can be used include chromophtalblue A3R, red iron oxide 3395, Neazopon Blue 807 (copper phthalocyanine-based dye), Brilliant Blue FCF, Fast Green FCF.

Anti-microbials which may be present include hexitidin, cetypyridiniumcloride (CPC), chlorhexidin (CHX), triclosan, stannous chloride, benzalkonium chloride, antimicrobial peptides (e.g. histatins), bactericins (e.g. nisin), antibiotics (e.g. tetracycline), aldehydes (e.g. glutaraldehyde), benzoic acid, salicylic acid, or there salts and derivative of such acids such as esters (e.g. p-hydroxy benzoate, parabens), and enzymes (e.g. lysozyme, oxidases).

Additives may be present in the following amounts: lower amount: 0 or at least 0.01 or at least 0.5 wt. %: upper amount: at most 10 or at most 8 or at most 5 wt. %; range: 0 to 10 or 0.1 to 8 or 1 to 5 wt. %: wt. % with respect to the whole composition.

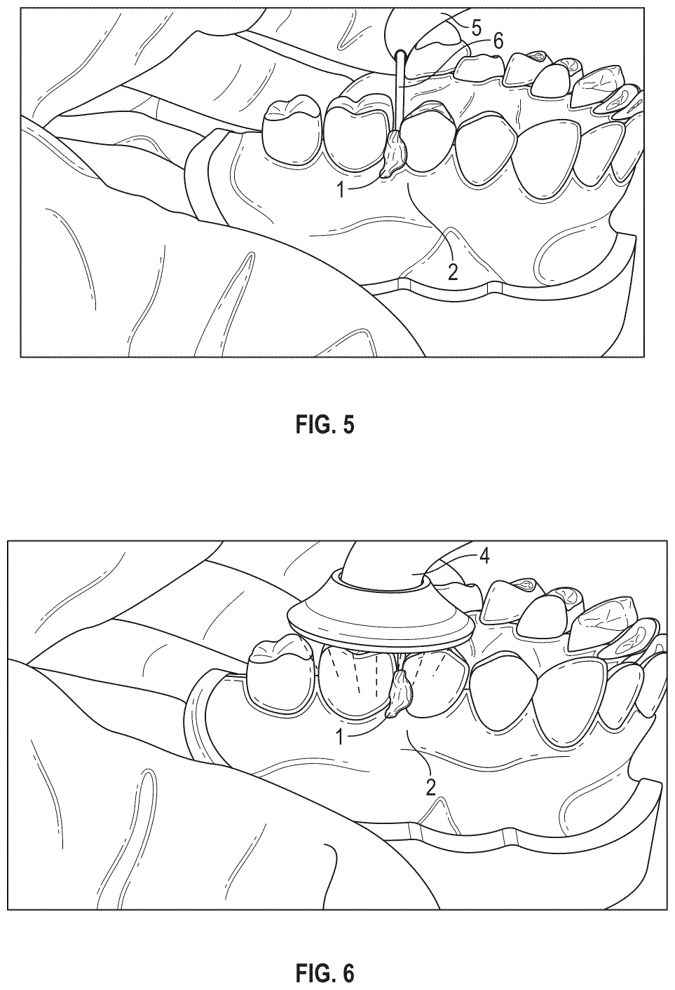

The composition described in the present text can typically be characterized by the following properties alone or in combination before hardening:
a) radiation curable with light having a wavelength in the range of 430 to 500 nm;
b) viscosity: less than 200 Pa*s, preferably in a range of 30 to 150 Pa*s at 23° C. and a shear rate of 50 s⁻¹;
c) water-uptake capacity: 25 to 450 wt. %;
d) extrusion force: 150 N or less, if the composition is dispensed from a container having a cannula with the dimension shown in FIG. 4 and using a piston as shown in FIGS. 5 and 6 of WO 2010/138433 A1:
e) being sticky to human tissue.

A combination of the features a) and b) or a), b) and c), or a), b), c) and d) or a), b), c), d) and e) can sometimes be preferred.

A composition with a viscosity in the above range may facilitate the application of the composition to the working area through a thin nozzle, e.g. by using a syringe-like device.

The pH value of the composition should not be too acidic or too basic in order to avoid irritation of the tissue to which the composition is applied.

The water-uptake capacity should not be too high as otherwise the composition will be expanding during the absorption process too much so that the composition after curing does not have a film-like appearance any longer.

If the composition is applied to the working area through a nozzle, the extrusion force during the application process should be not too high to enable an easy and uncomplicated application.

Ideally, the composition is sufficiently sticky to tissue, even if the tissue is wet. This further facilitates the application as the composition once applied to the tissue will not easily drip off the tissue or flow around.

The composition described in the present text can typically be characterized by the following properties alone or in combination after hardening:
a) tensile strength: 2 to 12 MPa according to DIN EN ISO 527-1:2012-06;
b) elongation at break: 40 to 200% according to DIN EN ISO 527-1:2012-06;
c) Shore hardness A: 40 to 92 according to ISO/DIN 53505 (2000-8);
d) depth of cure: 2 to 8 mm according to DIN EN ISO 6874 (2015).

A combination of the properties a) and b); or a), b) and c); or a), b), and d) is sometimes preferred.

A tensile strength in the above range can be beneficial as it facilitates the removal of the composition after curing in one piece.

An elongation at break in the above range can be beneficial as it makes the cured composition sufficiently elastic to adapt to the tissue or gum to be treated.

A Shore hardness A in the above range can be beneficial as it facilitates good removal properties especially in tight spaces.

The composition described in the present text may comprise the respective components in the following amounts: radiation curable component A1: 40 to 90 wt. %: photo-initiator: 0.1 to 5 wt. %; guanidyl containing polymer: 2 to 10 wt. %; carrageenan: 2 to 10 wt. %; softener: 0 to 20 wt. %; dye: 0 to 1 wt. %; filler: 0 to 15 wt. %; additives: 0 to 10 wt. %, wt. % with respect to the polymerizable composition.

A composition comprising the respective components in the following amounts is also suitable: radiation curable component A1: 45 to 85 wt. %; photo-initiator: 0.2 to 4 wt. %; guanidyl containing polymer: 3 to 9 wt. %; carrageenan: 3 to 9 wt. %; softener: 0.5 to 15 wt. %; dye: 0.005 to 0.8 wt. %; filler: 0.1 to 12 wt. %; additives: 0.1 to 8 wt. %; wt. % with respect to the polymerizable composition.

A composition comprising the respective components in the following amounts is also suitable: radiation curable component A1: 40 to 90 wt. %; radiation curable component A2: 1 to 25 wt. %; photo-initiator: 0.1 to 5 wt. %; guanidyl containing polymer: 2 to 10 wt. %; carrageenan: 2 to 10 wt. %; softener: 0 to 20 wt. %; dye: 0 to 1 wt. %; filler: 0 to 15 wt. %; additives: 0 to 10 wt. %, wt. % with respect to the polymerizable composition.

Further embodiments of the curable composition are described below:

### Embodiment 1

The composition described in the present text being characterized as follows:
the radiation curable component A1 not comprising a urethane moiety being present in an amount of 40 to 90 wt. %,
the urethan (meth)acrylate having a molecular weight (Mn) in the range of 200 to 1,000 g/mol, and being present in an amount of 0 to 35 wt. %,
the photo-initiator being present in an amount of 0.1 to 5 wt. %,
the guanidyl containing polymer being selected from polymers of the following formula and mixtures thereof,

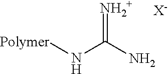

with X⁻ being selected from Cl⁻, Br⁻, I⁻, ½SO₄²⁻, NO₃⁻, CH₃COO⁻, C₃H₇COO⁻, and being present in an amount of 2 to 10 wt. %,
the carrageenan being selected from iota-carrageenan or lambda carrageenan, and being present in an amount of 2 to 10 wt. %,
the softener having a molecular weight (Mn) less than 1,000 g/mol, being selected from glycol, glycerine, ethylene glycol, poly(ethylene glycol), propylene glycol, poly(propylene glycol), copolymer(s) of ethylene glycol, propylene glycol and/or tetrahydrofuran and mixtures thereof, and being present in an amount of 0 to 20 wt. %,
the dye having a light absorption maximum in the range of 420 to 600 nm, and being present in an amount of 0 to 5 wt. %,
the filler being selected from silica, alumina, magnesia, titania, silicates and mixtures thereof, and being present in an amount of 1 to 10 wt. %, additives being present in an amount of 0 to 10 wt. %, wt. % with respect to the weight of the whole composition.

### Embodiment 2

The composition described in the present text being characterized as follows:
the radiation curable component A1 not comprising a urethane moiety being present in an amount of 40 to 90 wt. %,
the urethan (meth)acrylate having a molecular weight (Mn) in the range of 200 to 1,000 g/mol, and being present in an amount of 0 to 35 wt. %,
the photo-initiator being present in an amount of 0.1 to 5 wt. %,
the guanidyl containing polymer being selected from polymers of the following formula and mixtures thereof,

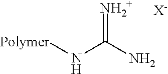

with X⁻ being selected from Cl⁻, Br⁻, I⁻, ½SO₄²⁻, NO₃⁻, CH₃COO⁻, C₃H₇COO⁻, and being present in an amount of 2 to 10 wt. %,
the carrageenan being selected from iota-carrageenan or lambda carrageenan, and being present in an amount of 2 to 10 wt. %,
the softener having a molecular weight (Mn) less than 1,000 g/mol, being selected from glycol, glycerine, ethylene glycol, poly(ethylene glycol), propylene glycol, poly(propylene glycol), copolymer(s) of ethylene glycol, propylene glycol and/or tetrahydrofuran and mixtures thereof, and being present in an amount of 1 to 20 wt. %,
the dye having a light absorption maximum in the range of 420 to 600 nm, and being present in an amount of 0.1 to 5 wt. %,
the filler being selected from silica, alumina, magnesia, titania, silicates and mixtures thereof, and being present in an amount of 1 to 10 wt. %, additives being present in an amount of 0 to 10 wt. %,
wt. % with respect to the weight of the whole composition.

### Embodiment 3

The composition described in the present text being characterized as follows:
the radiation curable component A1 not comprising a urethane moiety being present in an amount of 40 to 90 wt. %,
the urethan (meth)acrylate having a molecular weight (Mn) in the range of 200 to 1,000 g/mol, and being present in an amount of 1 to 35 wt. %,
the photo-initiator being present in an amount of 0.1 to 5 wt. %,
the guanidyl containing polymer being selected from polymers of the following formula and mixtures thereof,

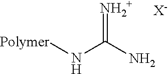

with X⁻ being selected from Cl⁻, Br⁻, I⁻, ½SO₄²⁻, NO₃⁻, CH₃COO⁻, C₃H₇COO⁻, and being present in an amount of 2 to 10 wt. %,
the carrageenan being selected from iota-carrageenan or lambda carrageenan, and being present in an amount of 2 to 10 wt. %,
the softener having a molecular weight (Mn) less than 1,000 g/mol, being selected from glycol, glycerine, ethylene glycol, poly(ethylene glycol), propylene glycol, poly(propylene glycol), copolymer(s) of ethylene glycol, propylene glycol and/or tetrahydrofuran and mixtures thereof, and being present in an amount of 1 to 20 wt. %,
the dye having a light absorption maximum in the range of 420 to 600 nm, and being present in an amount of 0.1 to 5 wt. %,
the filler being selected from silica, alumina, magnesia, titania, silicates and mixtures
thereof, and being present in an amount of 1 to 10 wt. %,
additives being present in an amount of 0 to 10 wt. %,
wt. % with respect to the weight of the whole composition.

The curable composition described in the present text does typically not comprise the following components alone or in combination: aluminium salts in an amount of 0.1 wt. % or more: polyacrylates or other superabsorbers being present in an amount of 1 wt. % or more: polymerizable components comprising acidic moieties in an amount of 1 wt. % or more: water in an amount of 0.1 wt. % or more: organic acid having a molecular weight of 45 to 250 g/mol, a pks value of 2 to 5, 1 to 3 carboxylic acid moieties, and being soluble in polyethylene glycol with a molecular weight of 400 g/mol in an amount of 1 wt. % or more, wt. % with respect to the whole composition.

The presence of alumina salts or polymerizable components comprising acidic moieties is typically not desired, as those substances might have an undesired impact on the healthy tissue to which the composition is applied.

The presence of polyacrylates or other superabsorbers is typically not desired, either, as those substances are often particulate and may have an undesired impact on the flow properties of the composition.

The composition described in the present text can be produced by mixing the respective components, in particular under safe-light conditions.

The composition is typically provided to the practitioner under hygienic conditions. During storage, the composition is typically packaged in a suitable packaging and delivery device.

One possibility to achieve this includes packing or storing the composition in a sealed container such as a capsule, cartridge, syringe or foil bag under hygienic conditions.

A suitable container may have a front end and a rear end, a piston movable in the container and a nozzle or cannula for delivering or dispensing the composition located in the container. The container has usually only one compartment or reservoir.

A suitable single-use container may have a volume in the range of 0.5 to 2 ml. This is the volume typically needed for a single sealing procedure. Such a container is typically used only once (e.g. disposable packing). If more than one sealing procedure is desired, the container may have a larger volume, e.g. in the range of 3 ml to 10 ml.

The composition can be dispensed out of the container by moving the piston in the direction of the nozzle. The piston can be moved either manually or with the aid of an application device or applier designed to receive the container (e.g. an application device having the design of a caulk gun).

According to one embodiment, the composition of the present text is stored in a one-compartment delivery device.

Examples of containers which can be used include compules, syringes and screw tubes. Containers of this kind are exemplified in more detail e.g. in U.S. Pat. No. 5,927,562 (Hammen et al), U.S. Pat. No. 5,893,714 (Arnold et al.) or U.S. Pat. No. 5,865,803 (Major).

It can be advantageous, if a container is used comprising a nozzle having a shape and size, which allows an easy and safe application of the composition to the soft dental tissue surrounding the tooth to be restored, also near the interdental region.

The smaller the diameter of the nozzle is, the easier the nozzle can be placed into the region between two teeth. However, a small diameter of the nozzle may result in an increase of the extrusion force needed for dispensing the composition out of the device. Thus, not all cannula sizes and diameters are equally suitable. A device with a nozzle or cannula having an external diameter in the range from 0.6 mm to 1.3 mm and an internal diameter in the range from 0.2 mm to 0.9 mm has been found to be particular useful.

It has been found that especially a container described in more detail in US 2011/151403 A1 (Pauser et al.) is useful for storing and dispensing the composition described in the present text.

The composition described in the present text is in particular useful for isolating or protecting tissue, including wet tissue.

A typical application process comprises the steps of
providing the composition described in the present text,
placing the composition in contact with soft tissue,
radiation curing the composition at least partially for a time period TI,
optionally moving or shaping the partially cured composition,
optionally radiation curing the composition for a time period T2, wherein T2>T1,
removing the composition from the soft tissue.

The placing of the composition can be accomplished by using an appropriate application system, including the application of the composition from a compule or syringe including those described in the present text.

The radiation curing of the composition can be accomplished by using an appropriate light source emitting light suitable for activating the photo-initiator of the composition. Suitable curing lights are commercially available and include e.g. Elipar™ DeepCure (3M Oral Care).

Upon curing, the adherence properties of the composition changes from sticky to non-sticky. As outlined above, a cured and thus non-sticky composition can be removed more easily compared to a sticky composition.

The removing of the partially or fully cured composition can be accomplished with any suitable device, including a pincer, a spatula or a probe.

Partially curing of the composition means that the composition is not completely cured, but e.g. that only the upper section or layer of the composition is cured. The upper section or layer may have a thickness of e.g. up to 80% or up to 60% of the thickness of the applied composition in a particular region.

Partially curing is typically accomplished by applying radiation only for a short time period, e.g. 1 to 5 sec. Fully curing of the composition can be accomplished by applying radiation for a longer time period, e.g. 10 to 30 sec. Depending on the overall thickness of the applied composition, the upper section or layer may have a thickness in the range of 0.1 to 2 mm.

If the composition is to be cured completely in one step, the time period T1 is typically in a range of 10 to 30 sec.

If the composition is to be cured in two steps, the time period T2 is typically longer than T1 (i.e. T2>T1 in sec.). Time period T2 is then typically in a range of 10 to 30 sec.

The curing steps may differ from each other by either the duration of the application of radiation, the intensity of the applied radiation or a combination of both.

The described process is visualized and exemplified in the attached figures in more detail.

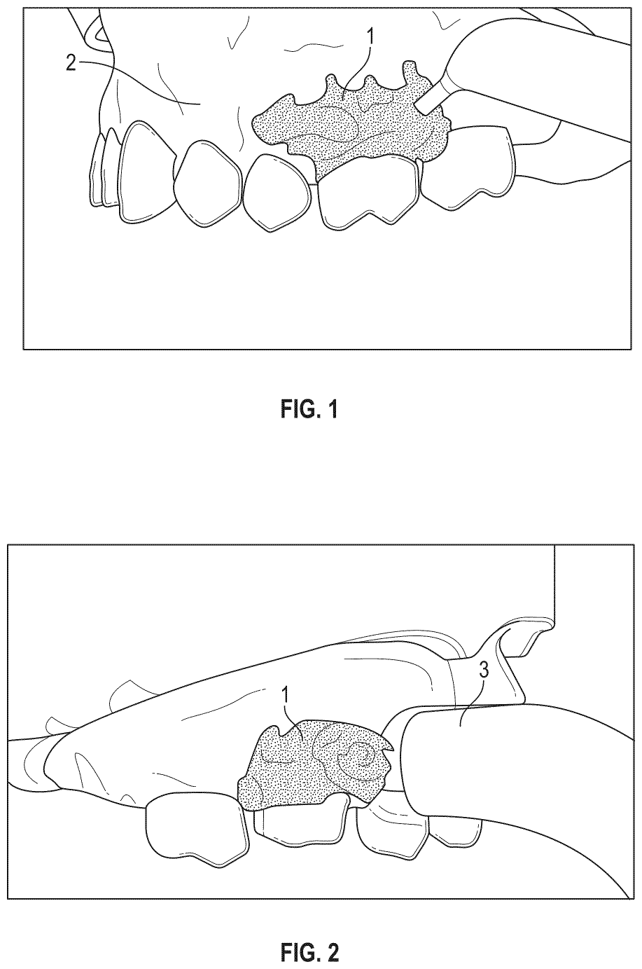

FIG. 1 shows the application of the composition 1 described in the present text to tissue 2 of a typodont model. Due to its inherent hydrophilic properties, the composition 1 sticks to the surface and does not flow or drop therefrom even if placed in the upper jaw region. The present moisture is essentially immediately absorbed by the composition.

In FIG. 2 the composition 1 is cured by applying light via a dental curing light 3. The consistency and/or mechanical properties of the composition change from pasty to rubber-elastic.

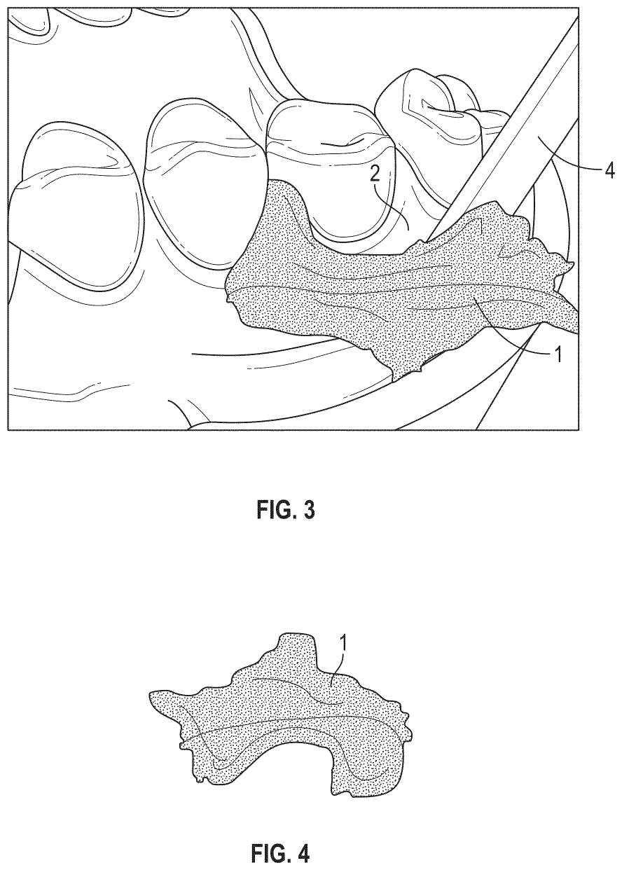

In FIG. 3 the removal of the light-cured composition 1 with a spatula 4 is shown. The composition can easily be removed from the tissue 2.

In FIG. 4 the removed cured composition 1 is shown. The composition 1 could be removed in one piece.

It is, however, also possible that the composition is cured stepwise, e.g. in two steps. Curing in two steps can be advantages for the practitioner as it make the system more flexible to use.

In FIG. 5 the composition 1 is applied to the oral tissue 2 through a thin nozzle 6 of a compule 5. Using a long and fine tip 6 makes is easy for the practitioner to reach narrow interproximal areas. If larger areas of the oral tissue 2 are to be covered other application systems with, e.g., wider tips or nozzles, can be used. Compared to the application of a dental retraction paste which is intended to widen the sulcus, the extrusion force needed for the application of the composition 1 described in the present text is rather low.

In FIG. 6 the composition 1 is partially cured using a dental curing light 4 with a wavelength in the range of 430 to 480 nm for about 5 sec. The time may vary depending on the formulation of the composition and on the type and the amount of photo-initiator used.

If a dye is present, the curing depth of the composition can be adjusted so that only the upper section or layer of the composition is cured. The lower section or layer being in contact with the tissue 2 remains in an uncured or partially cured stage.

The material has now a cured upper layer and an uncured or partially cured lower layer. This can be advantageous as the partially cured or uncured composition has better water-absorbing properties compared to the cured composition.

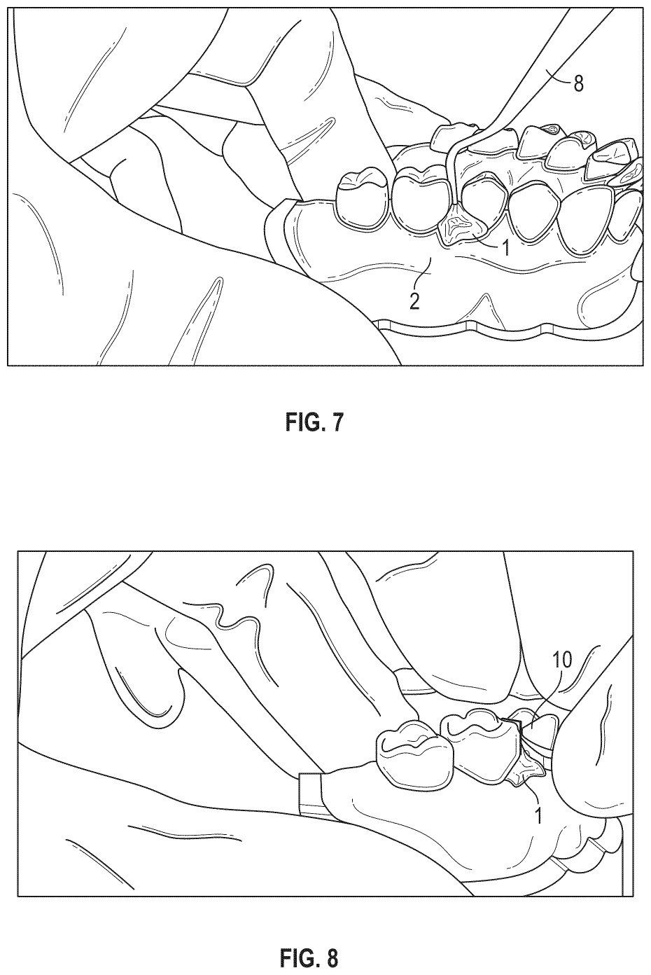

Further, in this stage the composition can be further modified, if desired, e.g. moved closer to the dental situation which needs to be restored or moved away from preparation lines or any other part of the operatory field which should not be covered by the composition. This is shown in FIG. 7 where the composition 1 is adapted with an instrument 8.

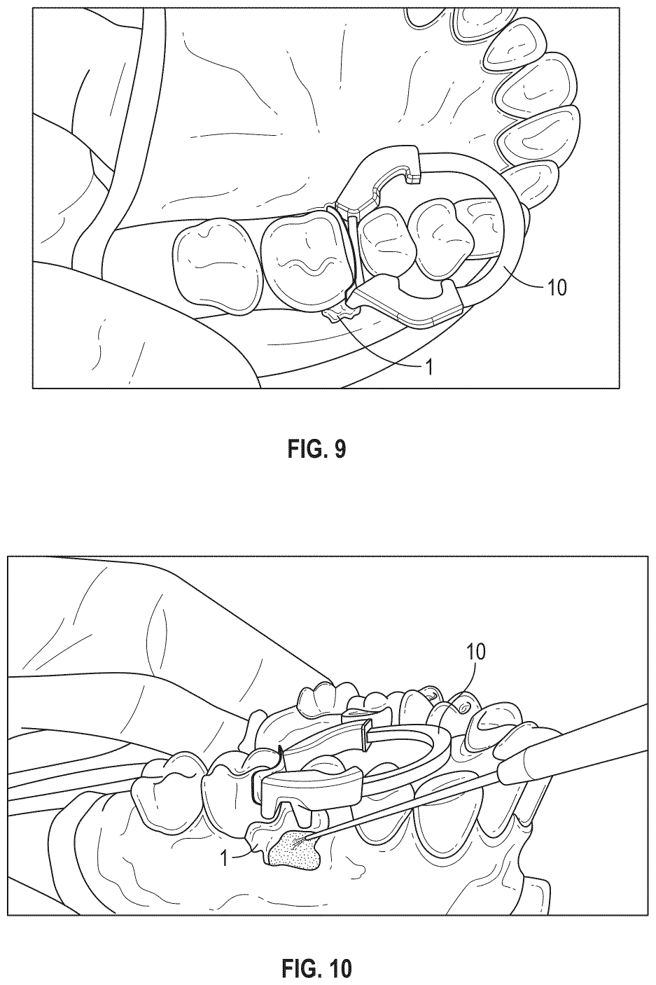

In a next step a dental matrix or template 10 is placed and fixed in the interproximal area to facilitate the restoration process of the tooth to be restored (FIG. 8; FIG. 9). The matrix 10 can be placed onto the composition 1 because its cured upper layer is not sticky or paste-like anymore and does not adhere or stick to the matrix itself or the further fixation means.

At the same time the lower uncured or partially cured section of the composition remains pasty and maintains its water absorbing properties, thus, preventing moisture from entering into areas of the matrix which are not fully mechanically sealed.

For demonstrating the water-absorbing properties of the partially cured composition coloured water is applied to the region of the tissue 2 which is covered by the composition 1 as shown in FIG. 10.

The cured upper section of the composition also prevents it from swelling towards the operatory field and only expands to the lower area. When doing so, if present, gaps, moisture producing areas or small wounds of the tissue are filled.

After the dental restoration procedure has been finished, the composition can be cured fully by conducting a further radiation-curing step, typically over a time period which is longer as the first radiation-curing step. e.g. for about 20 sec.

The fully cured material can then be removed in one or more pieces or rinsed with water, if desired.

Alternatively to the above-described process, the composition might also be placed after the matrix has been attached to the tooth.

The invention also relates to a kit of parts.

The kit of parts comprises the composition described in the present text and the following items alone or in combination: dental curing light: dental restoration material: dental adhesive: dental cement: dental matrix equipment.

Suitable dental curing lights include e.g. the Elipar™ Deep Cure LED curing light (3M Oral Care). Suitable dental restoration materials include e.g. Filtek™ Universal Restorative material (3M Oral Care). Suitable dental adhesives include e.g. Scotchbond™ Universal Adhesive (3M Oral Care) and Scotchbond™ Universal Plus Adhesive (3M Oral Care).

Dental adhesives are typically acidic dental composition with a rather low viscosity (e.g. 0.01 to 3 Pa*s at 23° C.). Dental adhesives directly interact with the enamel or dentin surface of a tooth. Dental adhesives are typically one-part compositions, are radiation-curable and comprise ethylenically unsaturated component(s) with acidic moiety, ethylenically unsaturated component(s) without acidic moiety, water, sensitizing agent(s), reducing agent(s) and additive(s). Examples of dental adhesives are described in US 2020/0069532 A1 (Thalacker et al.) and US 2017/0065495 A1 (Eckert et al.), US 2019/231494 A1 (Dittmann et al.). Suitable dental cements include RelyX™ Universal resin cement or RelyX™ Ultimate Adhesive dental resin cement (3M Oral Care). Dental matrix equipment or bands are pieces of metal or other material to support and to give form to the restoration during placement and hardening of the restorative material. Suitable include dental matrix equipment is commercially available, e.g. Palodent™ (DentsplySirona) or Omni-Matrix™ (Ultradent).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate the invention.

### EXAMPLES

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Water Uptake

0.10 g of the paste is placed in 0.50 g water (app. 20° C.). The paste stays in contact with water for 2.0 min. Then the paste is removed and the weight of the swollen paste is measured on a scale. The water uptake is calculated as percentage increase of the final weight in relation to the original weight (0.10 g).

### Water Contact Angel

If desired, the water contact angel can be determined as follows: a drop of a 10 wt. % ethanolic solution of the component to be tested is applied onto the surface of a dental mixing pad. The ethanolic solvent is evaporated to obtain a coated surface (approx. size: 4 cm²). On that surface a drop of water is placed and the development of the water-contact angle is analysed at 23° C. (Kruess Advance Software 1.13.1.31401). The average value obtained within 3 to 12s after placement of the drop is taken.

### Depth of Cure

If desired, the depth of cure can be determined in accordance with DIN EN ISO 6874:2015. The test is performed in a cylindrical metal form having a diameter of 4 mm and a length of 8 mm. Because of the elasticity of the resulting specimens instead of a measurement screw a ruler is used to measure the length of the specimen. The curing is done for 10s using a 3M Elipar™ DeepCure L with a LED emitting at 430-480 nm and 1480 mW/cm².

pH-Value

If desired, the pH value can be determined by using a wet pH sensitive paper.

### Viscosity

If desired, the viscosity can be measured using a Physica Rheometer MCR 302 device with a plate-plate system (diameter 20 mm) and a measuring gap width of 0.20 mm. The viscosity values (Pa*s) are being recorded at 23° C. for each shear rate (starting from 10 l/s to 100 l/s in steps of 10 l/s).

### Tensile Strength (TS) and Elongation at Break (EB)

If desired, the tensile strength and elongation of the compositions can be determined according to DIN EN ISO 527-1:2012-06. The tensile strength is given in MPa and the elongation in % of the original length. Tensile strength and elongation data were evaluated on a Zwick Z010 Universal testing machine, by tearing at least three I-shaped specimens of the following dimensions: Central unit: 10 mm×2 mm×2 mm; Overall length: 25 mm: Wider part width: 5 mm; Radius of rounded edges: R=10 mm on the central unit: 25 mm on the wider part.

The pastes were filled into a brass mould and section-wise light-cured on both sides at 23° C. using a Elipar™ DeepCure L with a LED emitting at 430-480 nm and 1480 mW/cm². The specimens were removed directly after the end of light-curing and put into an Otoflash device under Argon atmosphere for 1000 flashes. The measurements were performed at a Crosshead speed of 200 mm/min.

### Shore Hardness

If desired, the Shore A hardness of the compositions can be determined according to DIN 53505:2000-08 and measured 10 min after start of light curing. All samples are light-cured for 20 sec from both sides using a Elipar™ DeepCure L with a LED emitting at 430-480 nm and 1480 mW/cm². The specimens were directly after the end of light-curing, put into an Otoflash device under Argon atmosphere for 1000 flashes.

### Extrusion Force

If desired, the extrusion force can be measured using as testing device a Zwick Z020 machine (Zwick Roell Comp.). The testing device is equipped with a holder for containers and a small stamp to press against the piston inserted in the container and sealing the reservoir. The dimensions of the stamp corresponded to those used in commercially available single container dispensers (commercially available e.g. from 3M Oral Care). The feeding speed is set to 1.0 mm/s. The force is measured after the initial yield point was overcome (about 6-9 mm from starting point). The extrusion force is determined as an average value out of six individual measurements.

### Materials

TABLE 1

| Description | | Function |
|---|---|---|
| α-ω-methacrylated copolymer of | DIOL- | Radiation curable |
| ethylenoxide/THF; Mw about | MA | component |
| 6,300 g/mol; water-contact angle: | | |
| 54.8° | | |
| Diurethane dimethacrylate; 471 g/mol; | DUDMA | Radiation curable |
| ethylenoxide/THF; Mw about | | component |
| 6,300 g/mol; water-contact angle: 55.2° | | |
| Poly(ethylene glycol) dimethacrylate | PEG-MA | Radiation curable |
| Mw about 750 g/mol; | | component |
| water-contact angle: 2.5° | | |
| Polyethyleneglycol; Mw about | PEG400 | Softener |
| 400 g/mol | | |
| Guanylated polyethyleneimine | g-PEI | Water-absorbing |
| | | component |
| iota-carrageenan | i-CAR | Water-absorbing |
| | | component |
| Aerosil ™ 200 (Evonik) | Silica | Filler |
| Omnirad ™ 2022 (iGM Resins; | PO | Photo-initiator |
| Phosphine oxide | | |
| Camphorquinone | CQ | Photo-initiator |
| 4-Dimethylaminobenzoate | DMAB | Reducing agent |
| Lumogen ™ F yellow 083 | Dye | Perylene Dye |
| Tinuvin ™ 326 (BASF) | ABS | UV-Absorber |

### Preparation

All compositions described in Table I were prepared by homogenizing the respective components to a uniform paste using a planetary mixer with vacuum capabilities (Speedmixer DAC 600.1 VAZ-P).

### Compositions

The following compositions were prepared (Table 2):

TABLE 2

| Parts by | | | | | | | |
|---|---|---|---|---|---|---|---|
| Weight (pbw) | Ex. 1 | Ex. 2 | Ex. 3 | Ex. 4 | Ex. 5 | Ex. 6 | C. Ex. 1 |
| DIOL-MA | 54.27 | 54.27 | 54.27 | 54.27 | 54.27 | 54.33 | — |
| DUDMA | 17.00 | 17.00 | 17.00 | 17.00 | 17.00 | — | 17.00 |
| PEG-MA | — | — | — | — | — | 17.00 | 54.27 |
| PEG400 | 3.00 | 3.00 | 3.00 | — | 3.00 | 3.00 | 3.00 |
| g-PEI | 10.00 | 10.00 | 10.00 | 10.00 | 10.00 | 10.00 | 10.00 |
| i-CAR | 10.00 | 10.00 | 10.00 | 10.00 | 10.00 | 10.00 | 10.00 |
| Silica | 5.00 | 5.00 | 5.00 | 5.0 | 5.00 | 5.00 | 5.00 |
| PO | — | — | 0.4 | — | 0.4 | — | — |
| CQ | 0.40 | 0.40 | — | 0.40 | — | 0.40 | 0.40 |
| DMAB | 0.27 | 0.27 | — | 0.27 | — | 0.27 | 0.27 |
| Dye | 0.06 | — | 0.03 | — | — | — | — |
| ABS | — | — | — | — | 0.10 | — | — |

C. Ex. = comparative example

The properties of the compositions were further evaluated (Table 3).

TABLE 3

| | Ex. 1 | Ex. 2 | Ex. 3 | Ex. 4 | Ex. 5 | Ex. 6 | C. Ex. 1 |
|---|---|---|---|---|---|---|---|
| Tensile strength [MPa] | 5.99 | 6.99 | 6.96 | 6.16 | 9.37 | 2.69 | 7.21 |
| Elongation at break [%] | 110.6 | 120.7 | 88.0 | 98.8 | 96.9 | 70 | 23.1 |
| Depth of cure [mm] | 1.7 | 6.1 | 1.0 | 5.85 | 4.3 | 2.5 | 5.8 |
| Shore Hardness A | 82 | 86 | 90 | 89 | 91 | 71 | 95 |
| Water uptake [%] | 440 | 410 | 400 | 320 | 440 | 395 | 390 |
| Viscosity [Pa*s; 50 s⁻¹] | n.m. | 83 | 84 | 60 | 88 | 64 | 28 |

n.m. = not measured

All inventive examples showed high elongations at break and lower shore hardness indicating their elastomeric behaviour while maintaining sufficient water uptake. Inventive examples with the initiator PO showed lower cure depth than those containing the initiator system CQ/DMAB even at lower dye levels. This can be beneficial if only a partial curing is desired.

1. A composition for isolating tissue, the composition comprising
a radiation curable component A1, preferably being present in an amount of at least 40 wt. % with respect to the composition, the radiation curable component A1 comprising a polyether polyol spacer group having a molecular weight Mw of 1,000 to 20,000 g/mol, and at least two (meth)acrylate moieties, photo-initiator, guanidyl containing polymer, carrageenan, optionally softener,
optionally dye, optionally filler, and optionally additives,
the composition having preferably a viscosity of less than 200 Pa*s at 23° C. and a shear rate of 50s⁻¹.

2. The composition of claim 1 the ratio of radiation curable component A1 to guanidyl containing polymer being in a range of 10:1 to 5:1 with respect to weight.

3. The composition of claim 1, the ratio of guanidyl containing polymer to carrageenan being in a range of 2:1 to 1:2 with respect to weight.

4. The composition of claim 1 comprising the components in the following amounts:
radiation curable component A1: 40 to 90 wt. %,
photo-initiator: 0.1 to 5 wt. %,
guanidyl containing polymer: 2 to 10 wt. %,
carrageenan: 2 to 10 wt. %,
softener: 0 to 20 wt. %,
dye: 0 to 5 wt. %,
filler: 0 to 10 wt. %,
additives: 0 to 10 wt. %
wt. % with respect to the weight of the whole composition.

5. The composition according to claim 1 comprising a radiation curable component A1 and a radiation curable component A2 having a molecular weight in the range of 130 to 800 g/mol and being present in an amount lower than the amount of radiation curable component A1.

6. The composition of claim 1 comprising softener, the softener having a molecular weight Mw of less than 1,000 g/mol and being preferably selected from glycol, glycerine, ethylene glycol, poly(ethylene glycol), propylene glycol, poly(propylene glycol), and mixtures thereof.

7. The composition of claim 1 comprising dye, the dye being characterized by the following features alone or in combination:
having a light absorption maximum in the range of 420 to 600 nm;
comprising a perylene or naphtalimide moiety;
being a fluorescent dye.

8. The composition of claim 1 comprising in addition a urethan (meth)acrylate, preferably a urethan (meth)acrylate having a molecular weight (Mw) in the range of 200 to 1,000 g/mol.

9. The composition of claim 1 being characterized by the following features alone or in combination before hardening:
radiation curable with light having a wavelength in the range of 430 to 500 nm;
viscosity: less than 200 Pa*s at 23° C. and a shear rate of 50 s⁻¹;
pH value: 5 to 8 if brought in contact with water;
water-uptake capacity: 25 to 450 wt. %;
extrusion force: 150 N or less, if the composition is dispensed from a container having a cannula with the dimension shown in FIG. 4 and using a piston as shown in FIGS. 5 and 6 of WO 2010/138433 A1.

10. The composition of claim 1 being characterized by the following features alone or in combination after hardening:
tensile strength: 2 to 10 MPa according to ISO/DIN 53504 (2015-8);
elongation at break: 40 to 200% according to ISO/DIN 53504 (2015-8);
Shore hardness A: 30 to 90 according to ISO/DIN 53505 (2000-8);
depth of cure: 2 to 8 mm according to DIN EN ISO 6874 (2015).

11. The composition of claim 1 being characterized as follows:
the radiation curable component A1 not comprising a urethane moiety being present in an amount of 40 to 90 wt. %,
the urethan (meth)acrylate having a molecular weight in the range of 200 to 1,000 g/mol, and being present in an amount of 0 to 35 wt. %,
the photo-initiator being present in an amount of 0.1 to 5 wt. %,
the guanidyl containing polymer being selected from polymers of the following formula and mixtures thereof, 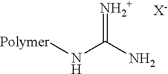
with X⁻ being selected from Cl⁻, Br⁻, I⁻, ½SO₄²⁻, NO₃⁻, CH₃COO⁻, C₃H₇COO⁻, and being present in an amount of 2 to 10 wt. %, and
the carrageenan being selected from iota-carrageenan or lambda carrageenan, and being present in an amount of 2 to 10 wt. %,
the softener having a molecular weight (Mn) less than 1,000 g/mol, and being selected from glycol, glycerine, ethylene glycol, poly(ethylene glycol), propylene glycol, poly(propylene glycol), copolymer(s) of ethylene glycol, propylene glycol and/or tetrahydrofuran and mixtures thereof, and being present in an amount of 0 to 20 wt. %,
the dye having a light absorption maximum in the range of 420 to 600 nm, and being present in an amount of 0.1 to 5 wt. %,
the filler being selected from silica, alumina, magnesia, titania, silicates and mixtures thereof, and being present in an amount of 1 to 10 wt. %,
additives being present in an amount of 0 to 10 wt. %,
wt. % with respect to the weight of the whole composition.

12. A packaging device containing the composition of claim 1, the packaging device comprising a dispensing nozzle and a reservoir for storing the composition, the reservoir having a volume of 0.5 to 10 ml, the dispensing nozzle having an internal diameter at the opening of 0.2 to 1 mm.

13. A process for temporarily isolating tissue, the process comprising the steps of providing the composition of claim 1
placing the composition in contact with tissue,
radiation curing the composition at least partially for a time period T1,
optionally moving or adjusting the partially cured composition,
optionally radiation curing the partially cured composition for a time period T2, wherein T2>T1, removing the composition from the tissue.

14. The process of claim 13, the curing being done in at least two steps, a first step for a time period T1 and a second step for a time period T2, the first and second step differing from each other by either the duration of radiation, or the intensity of radiation, or a combination of both.

15. A kit of parts comprising the composition described in claim 1 and the following items alone or in combination: dental curing light, dental restoration material, dental adhesive, dental cement, dental matrix equipment.
